(19) United States
(12) Patent Application Publication
(10) Pub. No.: US 2025/0360082 A1
(43) Pub. Date: Nov. 27, 2025

(54) ANTIFUNGAL SOLUTION, PREPARATION METHOD THEREFOR, AND USE THEREOF

(71) Applicant: SHANGHAI SKIN DISEASE HOSPITAL, Shanghai (CN)

(72) Inventors: Xiying WU, Shanghai (CN); Quangang ZHU, Shanghai (CN); Zhongjian CHEN, Shanghai (CN); Min SHEN, Shanghai (CN); Zongguang TAI, Shanghai (CN); Qin YU, Shanghai (CN); Jianping QI, Shanghai (CN); Wei WU, Shanghai (CN)

(73) Assignee: SHANGHAI SKIN DISEASE HOSPITAL, Shanghai (CN)

(21) Appl. No.: 18/872,635

(22) PCT Filed: Jun. 5, 2023
(86) PCT No.: PCT/CN2023/098300

§ 371 Date: Dec. 6, 2024

(30) Foreign Application Priority Data
Aug. 24, 2022 (CN) 202211021087.1

(51) Int. Cl. A61K 9/08; A61K 31/4174; A61K 31/4178; A61K 31/496; A61K 47/10; A61K 47/12; A61K 47/18; A61P 31/10

(52) U.S. Cl. CPC A61K 9/08; A61K 31/4174; A61K 31/4178; A61K 31/496; A61K 47/10; A61K 47/12; A61K 47/186; A61P 31/10

(57) ABSTRACT

An antifungal solution, a preparation method and use thereof are provided, belonging to the technical field of medicine. The antifungal solution includes an antifungal drug and a solvent as raw materials; where the antifungal drug and the solvent are at a mass ratio of (0.1-1):(18-318); and the solvent includes an ionic liquid, an alcohol organic solvent, and water. The ionic liquid has excellent solubility, skin penetration-promoting performance, and antifungal effect; the water can destroy an original polar network of the ionic liquid, forming smaller soluble ion groups, which greatly increase a miscibility window; the alcohol organic solvent can form a cosolvent with the ionic liquid and the water, thereby helping miscibility. The antifungal solution can ensure that the drug in a system is maintained in a dissolved state, thereby improving bioavailability and ensuring better therapeutic effects while reducing dosage.

### CROSS REFERENCE TO RELATED APPLICATIONS

The present application is the U.S. national phase of PCT Application No. PCT/CN2023/098300, filed on Jun. 5, 2023, which claims priority to Chinese Patent Application No. 202211021087.1 filed on Aug. 24, 2022, the disclosures of which are hereby incorporated herein by reference in their entireties.

### TECHNICAL FIELD

The present disclosure belongs to the technical field of medicine, and specifically relates to an antifungal solution, and a preparation method and use thereof.

### BACKGROUND

Tinea is a common superficial fungal infectious dermatosis that is widespread throughout the world. The most common pathogens of superficial fungal infections are dermatophytes, which have a high prevalence and are prone to recurrence (Yu et al., Mycoses 2020, 63, 1235-1243). Although tinea is not life-threatening, patients experience disproportionate amounts of itching, pain, and emotional distress, resulting in a greatly impaired quality of life.

Tinea may be treated with topical medications, oral medications, or a combination of both. Since some drugs may cause side effects such as pain, allergic reactions, and hepatotoxicity when being administered systemically, local medication is currently conducted to treat diseases in clinical practice (Shirsand et al., Int J Pharm Investig 2012, 2, 201-207). In recent years, it has made some progress using topical imidazole antifungal drugs that act on fungal cell membranes, including miconazole cream, 2% ketoconazole cream, clotrimazole cream, bifonazole cream, 1% luliconazole cream and other common imidazole products, in the treatment of tinea pedis (Crawford, BMJ clinical evidence 2009, 7, 1712; Chinese Working Group on Diagnosis and Treatment Guidelines for Tinea Manuum and Tinea Pedis, Chinese Journal of Mycology 2022, 17, 89-93). Unfortunately, studies have found that although topical imidazoles are more effective than placebo in treating fungal skin infections, their cure rates remain uncertain. Moreover, there is insufficient evidence to show that differences exist in the therapeutic effect of different drugs mentioned above (Crawford, BMJ clinical evidence 2009, 7, 1712). The unsatisfactory therapeutic effect of existing drugs in the treatment of tinea is closely related to drug dosage and dosage form. First of all, an excessively high concentration of the drug may cause adverse reactions or even cumulative poisoning, which can be fatal in severe cases due to the irritability to the skin of the drug itself. Secondly, an excessively low concentration of the drug may prevent it from reaching an effective therapeutic concentration at the lesion site, making it difficult to effectively exert the antifungal effect (Deng et al., Mater. Sci. Eng. C Mater. Biol. Appl. 2017, 78, 296-304; Jacobs et al., Drug Deliv. 2016, 23, 631-641). The above problems have greatly limited the application and promotion of imidazole antifungal drugs.

### SUMMARY

An objective of the present disclosure is to provide an antifungal solution, a preparation method and use thereof. In the present disclosure, the antifungal solution improves a therapeutic effect of tinea while reducing a dosage of the drug.

To achieve the above objective, the present disclosure provides the following technical solutions:

The present disclosure provides an antifungal solution, including an antifungal drug and a solvent as raw materials; where

the antifungal drug and the solvent are at a mass ratio of (0.1-1):(18-318); and
the solvent includes an ionic liquid, an alcohol organic solvent, and water.

In some embodiments, the ionic liquid, the alcohol organic solvent, and the water are at a mass ratio of (15-68):(3-30):(0.1-220).

In some embodiments, the antifungal drug includes an imidazole antifungal drug.

In some embodiments, the imidazole antifungal drug is selected from the group consisting of miconazole, ketoconazole, clotrimazole, bifonazole, and luliconazole.

In some embodiments, the ionic liquid includes a choline ionic liquid.

In some embodiments, the choline ionic liquid includes an aromatic acid-based choline ionic liquid.

In some embodiments, the alcohol organic solvent is one or more selected from the group consisting of ethanol, propylene glycol (PG), and polyethylene glycol (PEG).

The present disclosure further provides a preparation method of the antifungal solution described in the above solutions, including the following steps:
mixing the antifungal drug, the alcohol organic solvent, and the ionic liquid to allow heating to obtain a mixed solution; and
mixing the mixed solution with the water to obtain the antifungal solution.

In some embodiments, the heating is conducted at 60° C. to 90° C. for 1 h to 2 h.

The present disclosure further provides use of the antifungal solution described in the above solutions or an antifungal solution prepared by the preparation method described in the above solutions in preparation of an anti-tinea drug.

The antifungal solution provided in the present disclosure includes an antifungal drug and a solvent as raw materials; where the antifungal drug and the solvent are at a mass ratio of (0.1-1):(18-318); and the solvent includes an ionic liquid, an alcohol organic solvent, and water. The ionic liquid has excellent solubility, skin penetration-promoting performance, and antifungal effect; the water can dissolve anions through hydrogen bonds and can also dissolve aromatic rings in some cations, so as to disrupt an original polar network of the ionic liquid, forming smaller soluble ion groups, which greatly increase a miscibility window; the alcohol organic solvent can form a cosolvent with the ionic liquid and the water, thereby helping miscibility. The antifungal solution can ensure that the drug in a system is maintained in a dissolved state, thereby improving bioavailability and ensuring better therapeutic effects while reducing dosage. According to the data of examples, the antifungal solution improves a therapeutic effect of tinea while reducing a dosage of the antifungal drug, and reduces a recurrence rate, thereby overcoming the technical problem that a high dosage of topical imidazole antifungal drugs leads to a poor therapeutic effect.

### BRIEF DESCRIPTION OF THE DRAWINGS

To describe the technical solutions in examples of the present disclosure or in the prior art more clearly, the accompanying drawings required in the examples are briefly described below. Apparently, the accompanying drawings in the following description show merely some examples of the present disclosure, and other drawings can be derived from these accompanying drawings by those of ordinary skill in the art without creative efforts.

FIG. 1 shows an accumulated amount of the drug in the skin after treatment with different concentrations of ketoconazole antifungal solutions in Test Example 1;

FIG. 2 shows a flow chart of the Consolidated Standards of Reporting Trials (CONSORT) in Test Example 3; and

FIGS. 3A-3B show clinical symptom scores of patients in a KCZ-ILs group and a control group in Test Example 3 at each visit.

### DETAILED DESCRIPTION

The present disclosure provides an antifungal solution, including an antifungal drug and a solvent.

The present disclosure provides an antifungal solution, including an antifungal drug and

a solvent as raw materials; where the antifungal drug and the solvent are at a mass ratio of (0.1-1):(18-318); and

the solvent includes an ionic liquid, an alcohol organic solvent, and water.

In the present disclosure, all raw material components are commercially available products well known to those skilled in the art unless otherwise specified.

In the present disclosure, the antifungal drug and the solvent are at a mass ratio of (0.1-1):(18-318), preferably (0.2-0.8):(60-260), and more preferably (0.3-0.6):(100-200).

In the present disclosure, the ionic liquid, the alcohol organic solvent, and the water are at a mass ratio of preferably (15-68):(3-30):(0.1-220), more preferably (20-60):(10-28):(20-180), and most preferably (25-40):(15-25):(30-100).

In the present disclosure, the antifungal drug preferably includes an imidazole antifungal drug; the imidazole antifungal drug is preferably miconazole, ketoconazole, clotrimazole, bifonazole or luliconazole, and more preferably, miconazole, ketoconazole or bifonazole.

In the present disclosure, the antifungal drug acts on a fungal cell membrane to provide fungicidal efficacy.

In the present disclosure, the ionic liquid preferably includes a choline ionic liquid, and more preferably includes an aromatic acid-based choline ionic liquid; the aromatic acid-based choline ionic liquid preferably includes one or more of choline caffeate, choline gallate and choline enoate; the choline enoate preferably includes choline geranylate and/or choline 2,4-pentadienoate. When the ionic liquid includes two or more of the above specific options, there is no special limitation on a ratio of the above specific substances, any ratio is possible.

In the present disclosure, the ionic liquid exerts dissolving ability, skin penetration-promoting performance and antifungal effect.

In the present disclosure, the alcohol organic solvent preferably includes one or more of ethanol, PG and PEG, and more preferably includes the ethanol and/or PG. When the alcohol organic solvent includes two or more of the above specific options, there is no special limitation on a ratio of the above specific substances, any ratio is possible.

In the present disclosure, the alcohol organic solvent forms a cosolvent with the ionic liquid and water, such that the drug can reach a maximum solubility in the mixed solvent.

In the present disclosure, the water and the ionic liquid form an ionic liquid aqueous solution system, in which anions may be dissolved by interaction with hydrogen bonds and the aromatic rings of some cations may also be dissolved. In this way, an original polar network of the ionic liquid is disrupted, forming smaller soluble ion groups, which greatly increase a miscibility window.

In the present disclosure, the antifungal solution is preferably used externally, and specifically preferably: after cleaning and drying an affected area, the antifungal solution is fully applied to the affected area. The antifungal solution is preferably used twice a day separately in the morning and in the evening, with an application period of preferably 4 weeks.

The antifungal solution can ensure that the drug in a system is maintained in a dissolved state, thereby improving bioavailability and ensuring better therapeutic effect while reducing dosage. At the same time, the antifungal solution is comprehensive and fast-acting in terms of improving the skin lesion area, scaling, keratinization, and itching, thus solving the problem of single treatment of existing formulas. In addition, the antifungal solution is easy to use, has a light texture, desirable safety, and high patient compliance.

The present disclosure further provides a preparation method of the antifungal solution described in the above technical solutions, including the following steps:
mixing the antifungal drug, the alcohol organic solvent and the ionic liquid to allow heating to obtain a mixed solution; and
mixing the mixed solution with the water to obtain the antifungal solution.

In the present disclosure, the antifungal drug, the alcohol organic solvent and the ionic liquid are mixed to allow heating to obtain a mixed solution. There is no particular limitation on the mixing, and a process well known to those skilled in the art may be used. The heating is conducted at preferably 60° C. to 90° C., more preferably 70° C. for preferably 1 h to 2 h, more preferably 1.5 h. There is no particular limitation on a heating method, and a process well known to those skilled in the art may be used.

In the present disclosure, the heating is to cause hydrogen atom transfer between ions in the solution, such that the ions and solvent molecules form a complex, and energy released therefrom offsets the lattice energy of the salt to dissolve same. At the same time, the heating further promotes the loosening of the ionic liquid structure, reduces intramolecular and intermolecular interactions, and reduces the lattice energy of the ionic liquid, thereby promoting the formation of heterogeneous microstructures and liquid forms. In addition, the ionic liquid with longer terminal alkyl chains exhibits liquid crystal properties, enters an isotropic state upon heating, and is easily miscible.

In the present disclosure, the mixed solution is preferably allowed to stand and subjected to solid-liquid separation in sequence. The mixed solution is allowed to stand at preferably 20° C. to 40° C., more preferably 22° C. to 30° C., and most preferably at 24° C. to 26° C. for preferably 4 h to 48 h, more preferably 8 h to 36 h, and most preferably 12 h to 24 h. The solid-liquid separation is preferably conducted by centrifugation at preferably 5,000 rpm to 20,000 rpm, more preferably 8,000 rpm to 18,000 rpm, and most preferably 10,000 rpm to 15,000 rpm for preferably 2 min to 15 min, more preferably 4 min to 10 min, and most preferably 5 min to 7 min.

In the present disclosure, the effects of allowing to stand and centrifugation are to conduct solid-liquid separation on the drug to obtain a clarified mixed solution if the drug precipitates.

In the present disclosure, the mixed solution is mixed with the water to obtain the antifungal solution. The mixed solution and the water are at a volume ratio of preferably 1:(0.005-60), more preferably 1:(1-50), and most preferably 1:(5-30). There is no particular limitation on a mixing method, and a process well known to those skilled in the art may be used.

In the present disclosure, the preparation method of the antifungal solution has simple steps, can reduce costs, and is suitable for industrial production.

The present disclosure further provides use of the antifungal solution described in the above technical solutions or an antifungal solution prepared by the preparation method described in the above technical solutions in preparation of an anti-tinea drug.

In the present disclosure, the tinea preferably includes tinea manuum and tinea pedis, and more preferably the tinea pedis. There is no particular limitation on a manner of the use, and a process well known to those skilled in the art may be used.

In order to further illustrate the present disclosure, the antifungal solution, and the preparation method and the use thereof provided by the present disclosure are described in detail below with reference to the accompanying drawings and examples, but the accompanying drawings and the examples should not be construed as limiting the protection scope of the present disclosure.

### Example 1

0.5 g of ketoconazole, 15 g of PG, and 34 g of choline caffeate were mixed and heated at 60° C. for 2 h to obtain a mixed solution;
the mixed solution was allowed to stand at 24° C. for 12 h and then centrifuged at 10,000 rpm for 5 min to obtain a mixed solution and a precipitate; and
the resulting mixed solution in the last step was mixed with water at a volume ratio of 1:7 to obtain an antifungal solution containing 2.21 wt % of the ketoconazole.

### Example 2

0.5 g of ketoconazole, 15 g of PG, and 34 g of choline geranylate were mixed and heated at 90° C. for 1 h to obtain a mixed solution;
the mixed solution was allowed to stand at 24° C. for 12 h and then centrifuged at 10,000 rpm for 5 min to obtain a mixed solution and a precipitate; and
the resulting mixed solution in the last step was mixed with water at a volume ratio of 1:3, 1:6, 1:12, 1:24 and 1:48 to obtain ketoconazole-containing antifungal solutions KCZ-ILs, ½KCZ-ILs, ¼KCZ-ILs, ⅛KCZ-ILs and 1/16KCZ-ILs. The drug contents determined by HPLC were 4.72 mg/g, 2.36 mg/g, 1.18 mg/g, 0.59 mg/g and 0.30 mg/g, respectively.

### Example 3

0.5 g of bifonazole, 20 g of PG, and 56 g of choline gallate were mixed and heated at 60° C. for 2 h to obtain a mixed solution;
the mixed solution was allowed to stand at 24° C. for 24 h and then centrifuged at 10,000 rpm for 5 min to obtain a mixed solution and a precipitate; and
the resulting mixed solution in the last step was mixed with water at a volume ratio of 1:3, 1:6 and 1:12 to obtain bifonazole-containing antifungal solutions BIF-ILs, ½BIF-ILs and ¼BIF-ILs. The drug contents determined by HPLC were 5.98 mg/g, 2.99 mg/g and 1.50 mg/g, respectively.

### Use Example 1

Volunteer Zhou, female, 41 years old, was suffered from erosive tinea pedis for 1 year, with recurrent attacks and was in great pain. After treatment with the antifungal solution ½BIF-ILs provided in Example 3, which was applied onto the affected area twice a day, her skin lesion area was significantly reduced after one week, and the symptoms of scaling, keratinization and itching were all alleviated; after four weeks of medication, the clinical symptoms of tinea pedis were eliminated, and there has been no recurrence for more than 1 year.

### Use Example 2

Volunteer Wang, male, 50 years old, was suffered from keratotic tinea pedis for 5 years and had used medication repeatedly but with poor results. After treatment with the antifungal solution ½BIF-ILs provided in Example 3, which was applied onto the affected area twice a day, his skin lesion area was reduced, the scaling and keratinization were significantly reduced, and the itching symptoms disappeared after one week; the clinical evaluation was effective after four weeks of medication; the clinical symptoms were basically eliminated after continuing the medication for another 2 weeks, and there has been no recurrence for more than half a year.

### Test Example 1

The accumulation of ketoconazole in the skin was tested in a rat treated with different concentrations of ketoconazole in antifungal solutions:

Experimental group: the antifungal solutions KCZ-ILs, ½KCZ-ILs, ¼KCZ-ILs, ⅛KCZ-ILs and 1/16KCZ-ILs obtained in Example 2 were added into Franz diffusion cells for excised rat skin for 6 h, the skin was taken off, cleaned, then cut into pieces, followed by immersed in 50% methanol solution, which was allowed to stand overnight, and centrifuged to obtain a supernatant containing the drug, and a drug content in the skin was determined by HPLC.

Control group: the experimental procedures were the same as above, except that the antifungal solution was replaced with an equal amount of commercially available 2% ketoconazole cream.

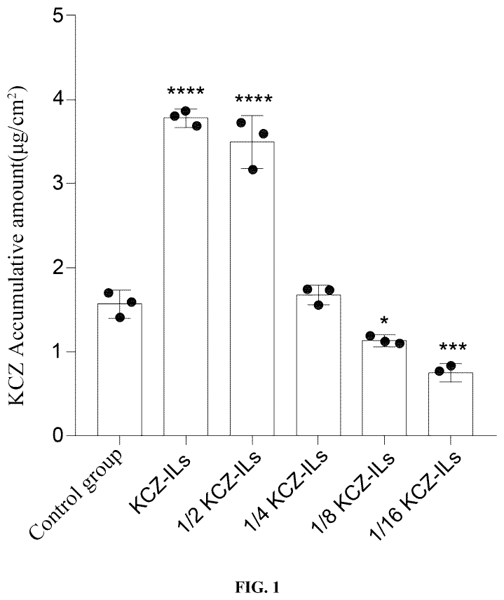

The results were shown in FIG. 1.

As shown in FIG. 1, the accumulation of ketoconazole in the skin in KCZ-ILs and ½KCZ-ILs groups was more than 2 times that of the 2% ketoconazole cream in the control group; the accumulation effect of the ¼KCZ-ILs group was equivalent to that of the control group; and the accumulation effects of the ⅛KCZ-ILs and 1/16KCZ-ILs groups were both inferior to that of the control group. Therefore, by a ketoconazole antifungal solution with a concentration of 1.18 mg/g or more, that is, a concentration of greater than 0.118%, an accumulation amount of drug in the skin that is more than that of the commercially available 2% ketoconazole cream could be achieved.

### Test Example 2

The accumulation of bifonazole in the skin was tested in a rat treated with different concentrations of bifonazole in antifungal solutions:

Experimental group: the antifungal solutions BIF-ILs, ½BIF-ILs, and ¼BIF-ILs obtained in Example 2 were added into Franz diffusion cells for excised rat skinfor 6 h, the skin was taken off, cleaned, then cut into pieces, followed by immersed in 50% methanol solution, which was allowed to stand overnight, and centrifuged to obtain a supernatant containing the drug, and a drug content in the skin was determined by HPLC.

Control group: the experimental procedures were the same as above, except that the antifungal solution was replaced with an equal amount of commercially available 1% bifonazole cream.

The results showed that the accumulation of BIF-ILs in the skin (3.78±0.09 μg/cm²) was 2 times that of the control group (1.85±0.19 μg/cm²); the accumulation effect of the ½BIF-ILs group (1.89±0.13 μg/cm²) was equivalent to that of the bifonazole cream in control group. Therefore, by a bifonazole antifungal solution with a concentration of 2.99 mg/g or more, that is, a concentration of greater than 0.299%, an accumulation amount of drug in the skin that is more than that of the commercially available 1% bifonazole cream could be achieved.

### Test Example 3

### (1) Clinical Data

All cases were collected from patients with tinea pedis who visited the general dermatology outpatient clinic of Shanghai Dermatology Hospital from August 2021 to November 2021. According to the clinical diagnostic criteria, 36 patients with tinea pedis who were eligible for inclusion and had positive fungal microscopic examination of the skin lesions were divided into an experimental group (KCZ-ILs group, 18 cases, 17 of which completed the trial) and a control group (ketoconazole cream group, 18 cases, 16 of which completed the trial) by simple ranking randomization, where the patients were aged 20 to 65 years old.

### (2) Drugs

Experimental group: ketoconazole antifungal solution KCZ-ILs prepared in Example 2.

Control group: ketoconazole cream (10 g: 0.2 g, namely 20 mg/g, Xi'an Janssen Pharmaceutical Co., Ltd., sFDA approval number H20043171).

### (3) Therapies

The affected area was cleaned, kept dry and comfortable, then an appropriate amount of the drug was applied onto the affected area, twice a day for 4 consecutive weeks.

### (4) Observation Indicators and Evaluation Criteria for Therapy Effect

Clinical total symptom score (TSS) records and mycological analysis of skin samples were conducted at week 0 (baseline), 1, 2, 3, 4, and 8 (endpoint). TSS and mycological results were recorded at weekly follow-up visits during the first 4 weeks. After 4 weeks of drug withdrawal, patients were followed up for disease recurrence. The main therapeutic effect indicator was clinical therapeutic effect after 4 weeks of treatment, where clinical therapeutic effect was defined as a negative mycological result and a decrease in the TSS of at least 60% from baseline. Secondary therapeutic effect endpoints for evaluating disease recurrence included the proportion of clinical responders at week 8 and fungal recurrence rates at weeks 2, 3, 4, and 8. In addition, changes in TSS, mycological eradication rate, and adherence were further analyzed at each visit. Drug safety was evaluated in aspects of physical examination, vital signs, and adverse events, particularly irritation reactions such as erythema, desquamation, papules, and vesicles.

### i) Clinical TSS Indicators and Criteria

The clinical TSS was calculated based on the scores of skin lesion area, maceration degree, erosion area, exudation degree, scaling, keratinization and itching, with the severity of each symptom was divided into 4 levels (from 0=no symptom to 3=the most severe symptom), and criteria were shown in Table 1.

TABLE 1
Scoring criteria for clinical symptoms

| Scoring item | 0 | 1 | 2 | 3 |
|---|---|---|---|---|
| Skin lesion | None | Mild (<2.5 | Medium (2.5-5 | Severe (>5 |
| area | | cm²) | cm²) | cm²) |
| Maceration | None | Mild (1 | Medium (2 toe | Severe (3 toe |
| degree | | toe gap) | gaps) | gaps) |
| Erosion area | None | Mild (<2.5 | Medium (2.5-5 | Severe (>5 |
| | | cm²) | cm²) | cm²) |
| Exudation | None | Mild (surface | Medium (exudation | Severe (with |
| degree | | exudation) | with serous scab) | fluid) |
| Scaling | None | Mild (<2.5 | Medium (2.5-5 | Severe (>5 |
| | | cm²) | cm²) | cm²) |
| Kerati- | None | Mild (<2.5 | Medium (2.5-5 | Severe (>5 |
| nization | | cm²) | cm²) | cm²) |
| Itching | None | Mild | Medium | Severe |
| | | (occasional, | (frequent, | (frequent/ |
| | | no need | slightly | persistent, |
| | | to scratch) | scratchy) | unbearable) |

### ii) Mycological Examination

Skin scrapings were collected from the infected sites and tested for mycology, with potassium hydroxide (KOH) testing used as a proxy for mycological results.

iii) Indicators and Criteria for Therapy Effect Evaluation

The therapeutic effect was evaluated based on the results of clinical TSS and fungal examination, including cured, markedly effective, effective, and ineffective.

$$Efficacy index ( \% ) = \frac{pre - treatment TSS - post - treatment TSS}{post - treatment TSS} \times 100 \%$$

Index of therapeutic effect (%)=pre-treatment TSS-pos-treatment TSS/pos-treatment TSS
cured: TSS=100%, clinical symptoms disappear, skin lesions completely disappear, and fungal microscopy being negative;
markedly effective: 60%≤TSS≤99%, symptoms significantly relieved, fungal microscopy being negative or showing extremely small amount of hyphae;
effective: 20%≤TSS≤59%, symptoms alleviated, fungal microscopy possibly being positive;
ineffective: TSS<20%, no significant change in symptoms, and fungal microscopy being positive.

$$Clinical effective rate = \frac{( Number of cure cases + Number of marked effectiveness cases )}{Total cases} \times 100 \%$$

$$Clinical effective rate = Number of cure cases + Number of markedly effective cases \text{} / Total cases * 100 \%$$

### iv) Indicators for Safety Evaluation

The occurrence of adverse reactions during the patient's treatment was observed, and local skin irritation reactions were particularly focused.

### (5) Analysis of Subject Data

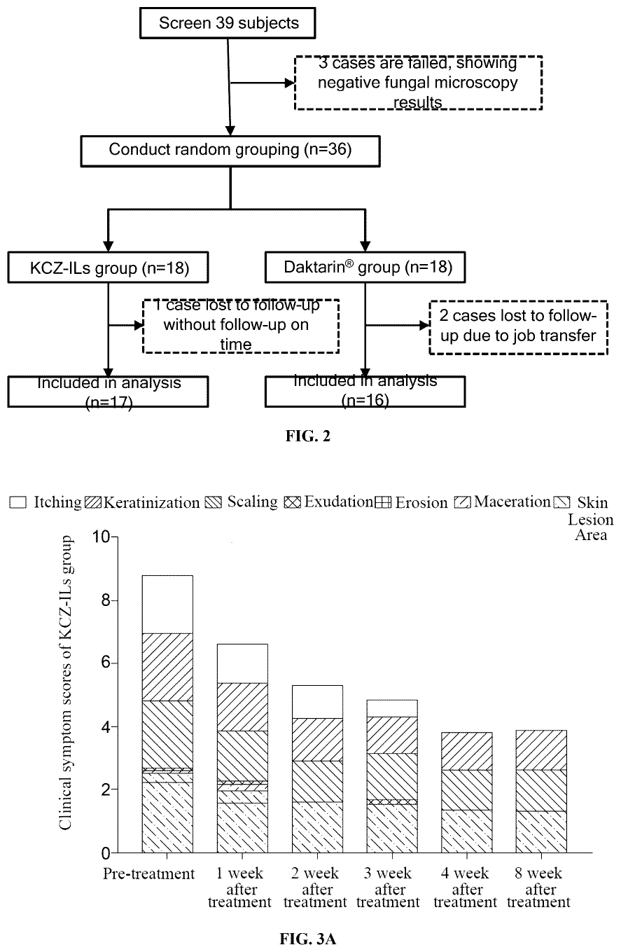

A total of 39 patients were screened, of whom 36 met the inclusion and exclusion criteria. 3 patients were lost to follow-up during the study period due to job changes or unpunctuality. Of the 33 fully evaluable patients, 17 cases were treated with KCZ-ILs and 16 cases with Daktarin®. The CONSORT diagram of the participants was shown in FIG. 2. At the end of treatment, the compliance rates with the trial regimen were 96.4% and 98.4% in the KCZ-ILs group and the Daktarin® group, respectively.

TABLE 2
Demographic and clinical characteristics of patients at baseline

| | KCZ-ILs | | Control | |
|---|---|---|---|---|
| Variables | (n = 17) | | (n = 16) | |
| Age, mean (SD), years | 48.88 | (9.46) | 49.13 | (8.15) |
| Gender, No. (%) | | | | |
| Female | 8 | (47.06) | 8 | (50.00) |
| Male | 9 | (52.94) | 8 | (50.00) |
| mean (SD) of body mass | 24.09 | (2.58) | 24.47 | (2.93) |
| index, in kg/m² | | | | |
| Body mass index grouping, No. (%) | | | | |
| <25 kg/m² | 10 | (58.82) | 11 | (68.75) |
| 25~30 kg/m² | 6 | (35.29) | 4 | (25.00) |
| >30 kg/m² | 1 | (5.88) | 1 | (6.25) |
| mean (SD) of TSS | 8.76 | (2.14) | 9.44 | (3.18) |
| Complication*, No. (%) | 2 | (11.76) | 2 | (12.50) |

*Complication: hypertension (n = 2, 1 case in each group), diabetes (n = 1, in KCZ-ILs group), or both complications (n = 1, in control group).

As shown in Table 2, there were 17 cases in the experimental group, including 9 males and 8 females, aged 31-59 years old, and the clinical TSS was 8.76±2.14; and there were 8 males and 8 females in the control group, aged 32-59 years old, and the clinical TSS was 9.44±3.18. A small number of patients (4/33, 12%) had a combination of hypertension (2/33, 6%), diabetes (1/33, 3%), or both (1/33, 3%). Except for body mass index, There was no significant difference in characteristics at baseline between the two groups of patients (P>0.05).

### (6) Clinical TSS

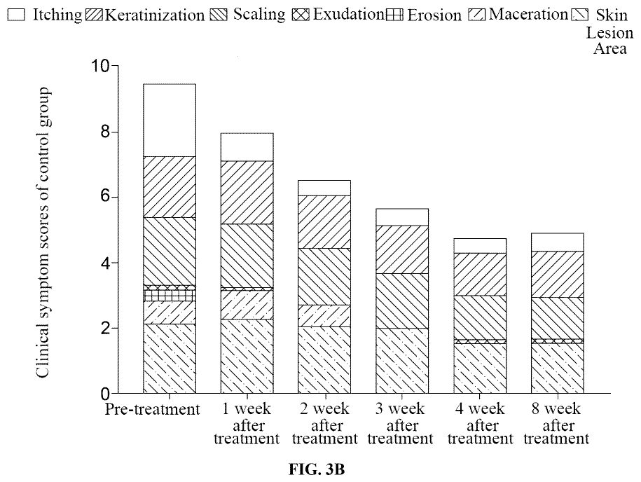

After 4 weeks of treatment, the TSS change was −4.94 (±2.51) [p<0.001] in the KCZ-ILs group and −4.44 (±2.34) [p<0.001] in the Daktarin® group. Symptoms were improved after just one week of treatment and the effects lasted throughout the study period. There was a statistically significant difference in TSS between the two groups (F=58.58, P<0.001), mainly at the 2nd and 3rd weeks. FIGS. 3A-3B showed clinical symptom scores of patients in a KCZ-ILs group and a control group at each visit.

As shown in FIGS. 3A-3B, KCZ-ILs showed significant improvements in skin lesion area, scaling, keratinization and itching, while Daktarin® tended to reduce scaling.

### (7) Fungal Clearance Rate

TABLE 3
Mycological examination results

| Variables | KCZ-ILs (n = 17) | | Control (n = 16) | | P value^{a} |
|---|---|---|---|---|---|
| Baseline, No. (%) | | | | | |
| Positive | 17 | (100.00) | 16 | (100.00) | 1.000 |
| Negative | 0 | (0) | 0 | (0) | |
| Week 1, No. (%) | | | | | |
| Positive | 10 | (58.82) | 8 | (50.00) | 0.732 |
| Negative | 7 | (41.18) | 8 | (50.00) | |
| P value^{b} | 0.002 | | 0.152 | | |
| Week 2, No. (%) | | | | | |
| Positive | 6 | (35.29) | 8 | (50.00) | 0.491 |
| Negative | 11 | (64.71) | 8 | (50.00) | |
| P value^{b} | 0.031 | | 0.152 | | |
| Negative to positive^{c} | 2 | (28.57) | 3 | (37.50) | 0.325 |
| Week 3, No. (%) | | | | | |
| Positive | 6 | (35.29) | 8 | (50.00) | 0.491 |
| Negative | 11 | (64.71) | 8 | (50.00) | |
| P value^{b} | 0.031 | | 0.152 | | |
| Negative to positive^{c} | 1 | (9.09) | 3 | (37.50) | <0.001 |
| Week 4, No. (%) | | | | | |
| Positive | 4 | (23.53) | 7 | (43.75) | 0.282 |
| Negative | 13 | (76.47) | 9 | (56.25) | |
| P value^{b} | 0.125 | | 0.230 | | |
| Negative to positive^{c} | 2 | (18.18) | 3 | (37.50) | 0.008 |
| Week 8, No. (%) | | | | | |
| Positive | 7 | (41.18) | 8 | (50.00) | 0.732 |
| Negative | 10 | (58.82) | 8 | (50.00) | |
| P value^{b} | 0.016 | | 0.152 | | |
| Negative to positive^{c} | 4 | (30.77) | 2 | (22.22) | 0.216 |

^{a}differences between the KCZ-ILS group and the control group.
^{b}differences in the KCZ-ILS group or control group compared with baseline.
^{c}a proportion of patients who turned from negative to positive was a proportion of new positive cases to a total number of negative cases in the previous period.

As shown in Table 3, the therapeutic effect of the KCZ-ILs group increased over time, with only 4 positive patients at the end of treatment and a fungal clearance rate of 76.47%. The fungal clearance rate of the Daktarin® group was 50.00% (8/16 cases) at weeks 1-3 and 56.25% (9/16 cases) at week 4. During the entire trial period, 6 (35.29%) fungus-negative cases in KCZ-ILs group became positive, compared with 11 of 16 fungus-negative cases in the control group (68.75%).

### (8) Overall Therapeutic Effect

TABLE 4
Overall therapeutic effect of two groups at week 4 and week 8

| | | |
|---|---|---|
| Overall therapeutic effect | KCZ-ILs (n = 17) | Control (n = 16) |

| | | | | |
|---|---|---|---|---|
| Week 4, No. (%) | | | | |
| Cured | 1 | (5.88) | 1 | (6.25) |
| Markedly effective | 7 | (41.18) | 3 | (18.75) |
| Effective | 9 | (52.94) | 9 | (56.25) |
| Ineffective | 0 | (0) | 3 | (18.75) |
| Clinical effective | 8 | (47.06) | 4 | (25.00) |
| Week 8, No. (%) | | | | |
| Cured | 2 | (11.76) | 2 | (12.50) |
| Markedly effective | 5 | (29.41) | 3 | (18.75) |
| Effective | 9 | (52.94) | 10 | (62.50) |
| Ineffective | 1 | (5.88) | 1 | (6.25) |
| Clinical effective | 7 | (41.17) | 5 | (31.25) |

As shown in Table 4, among the patients in the KCZ-ILs group, approximately 53% cases showed effective, 41% cases showed markedly effective, and 6% cases showed cured; while among the patients in the control group, there were 19%, 56%, 19% and 6% cases showing ineffective, effective, markedly effective and cured, respectively. The primary therapeutic effect outcome was the proportion of patients showing clinical effective (cured and markedly effective). At the end of treatment, the clinical effective rate of the KCZ-ILs group was 47.06%, while that of the Daktarin® group was only 25.00%. At week 4, the therapeutic effect of the KCZ-ILs group was superior to that of the Control group, and the difference was statistically significant (P=0.010). 4 weeks after the end of treatment, the clinical effective rate of the KCZ-ILs group was 41.17%, while that of the control group was 31.25%.

### (9) Adverse Reactions

No adverse reactions were observed in the experimental group, and 1 case (6.25%) was observed in the control group. The main symptom of adverse reaction was transient mild erythema at the lesion one week after applying the drug, which did not affect the continued treatment and therapeutic effect evaluation. There was no statistically significant difference in terms of adverse reactions between the two groups.

In this clinical trial, the therapeutic effect and safety of a ketoconazole-loading antifungal solution in the treatment of patients with tinea pedis were evaluated. KCZ-ILs (KCZ, 4.72 mg/g) was significantly superior to Daktarin® (KCZ, 20 mg/g) in terms of clinical improvement and prevention of recurrence. Furthermore, KCZ-ILs was well tolerated without any adverse events, while there was one patient in the control group reported transient mild erythema. In conclusion, ILs loaded with only ¼ KCZ dose of the control preparation showed better therapeutic effect and safety in the treatment of tinea pedis, which created new opportunities for the treatment of skin diseases caused by fungal infections and was worthy of clinical application.

According to the above examples, the antifungal solution provided in the present disclosure improves a therapeutic effect to tinea while reducing a dosage of the drug. At the same time, the antifungal solution provided in the present disclosure is comprehensive and fast-acting in terms of improving the skin lesion area, scaling, keratinization, and itching, thus solving the problem of single treatment of existing formulas. In addition, the antifungal solution provided in the present disclosure is easy to use, light in texture, good in safety, and high in patient compliance.

Although the present disclosure is described in detail in conjunction with the foregoing examples, they are only a part of, not all of, the examples of the present disclosure. Other examples can be obtained based on these examples without creative efforts, and all of these examples shall fall within the protection scope of the present disclosure.

1. An antifungal solution, comprising an antifungal drug and a solvent as raw materials; wherein
the antifungal drug and the solvent are at a mass ratio of (0.1-1):(18-318); and
the solvent comprises an ionic liquid, an alcohol organic solvent, and water.

2. The antifungal solution according to claim 1, wherein the ionic liquid, the alcohol organic solvent, and the water are at a mass ratio of (15-68):(3-30):(0.1-220).

3. The antifungal solution according to claim 1, wherein the antifungal drug comprises an imidazole antifungal drug.

4. The antifungal solution according to claim 3, wherein the imidazole antifungal drug is selected from the group consisting of miconazole, ketoconazole, clotrimazole, bifonazole and luliconazole.

5. The antifungal solution according to claim 1, wherein the ionic liquid comprises a choline ionic liquid.

6. The antifungal solution according to claim 5, wherein the choline ionic liquid comprises an aromatic acid-based choline ionic liquid.

7. The antifungal solution according to claim 1, wherein the alcohol organic solvent is one or more selected from the group consisting of ethanol, propylene glycol (PG) and polyethylene glycol (PEG).

8. A method for preparing the antifungal solution according to claim 1, comprising the following steps:
mixing the antifungal drug, the alcohol organic solvent, and the ionic liquid to allow heating to obtain a mixed solution; and
mixing the mixed solution with the water to obtain the antifungal solution.

9. The method according to claim 8, wherein the heating is conducted at 60° C. to 90° C. for 1 h to 2 h.

10. A method for preparing an anti-tinea drug, comprising using the antifungal solution according to claim 1 as a raw material.

11. A method for preparing an anti-tinea drug, comprising using the antifungal solution prepared by the method according to claim 8 as a raw material.
